(19) United States
(12) Patent Application Publication
(10) Pub. No.: US 2014/0347185 A1
(43) Pub. Date: Nov. 27, 2014

(54) HAND HYGIENE NETWORK SYSTEM

(71) Applicant: Smith Intellectual Property, LLC, Greencastle, IN (US)

(72) Inventors: Judson Smith, Greencastle, IN (US); Bryan D. Smith, Phoenix, AZ (US); Garrett J. Smith, Chicago, IL (US)

(21) Appl. No.: 14/453,244

(22) Filed: Aug. 6, 2014

Related U.S. Application Data
(63) Continuation of application No. 14/371,257, filed on Jul. 9, 2014.
(60) Provisional application No. 61/584,418, filed on Jan. 9, 2012.

(51) Int. Cl. G08B 23/00; A61L 2/00; A47K 5/12

(52) U.S. Cl. CPC A61L 2/0088; A47K 5/1217
USPC 340/573.1; 222/180; 222/52

(57) ABSTRACT

In a health care facility, within the immediate patient care zone, a method of providing a networked system for hand hygiene. At least one (1) health care worker (HCW) has the means to perform at least one (1) event of the “World Health Organization's (WHO) 5 Moments Hand Hygiene” protocol events utilizing a movable hand hygiene dispenser and/or fixedly mounted hand hygiene dispenser positioned within the patient care zone. The HCW's “WHO'S Hand Hygiene” events are monitored by a wireless network of transceivers, located within the patient care zone, which can communicate information between one another and/or to the health care facility's computer memory and processing center for the purposes of HCW's alerts, process steps, evaluation, reporting, etc., of the HCW's hand hygiene event activity and his/her compliance relative to the “WHO'S 5 Moments Hand Hygiene” events protocol.

### CROSS REFERENCE TO RELATED APPLICATION

This application claims the benefit of priority to U.S. Provisional Patent Application Ser. No. 61/584,418, filed Jan. 9, 2012, entitled 5 MOMENTS HAND HYGIENE NETWORK SYSTEM, incorporated herein by reference.

### FIELD OF THE INVENTION

The present invention pertains to methods and apparatus used to maintain hygienic conditions, and in particular to hand washing dispenser with electronic monitoring.

### BACKGROUND

The World Health Organization's (WHO) Guidelines on Hand Hygiene in Health Care reports that: “Health Care—Associated Infections (HCAI) is a major problem for patient safety and its surveillance and prevention must be a priority for settings and institutions committed to making health care safer.”

The report further states: “The impact of HCAI implies prolonged hospital stay, long-term disability, increased resistance of microorganisms to antimicrobials, massive additional financial burden, high costs for patients and their families, and excess deaths. Although the risk of acquiring HCAI is universal and pervades every health-care facility and system around the world, the global burden is unknown because of the difficulty of gathering reliable diagnostic data. Overall estimates indicate that more than 1.4 million patients worldwide in developed and developing countries are affected at any time.”

The WHO report states that there are “5 Moments for Hand Hygiene” at which a Health Care Worker (HCW) should comply with hand hygiene protocols, when providing safe care for patients. The following 5 Moments for Hand Hygiene are performed in the patient care zone and critical “point-of-care” sites:
Moment 1. Before touching a patient
Moment 2. Before a clean/aseptic procedure
Moment 3. After body fluid exposure risk
Moment 4. After touching a patient
Moment 5. After touching patient surroundings

The WHO regards a “point-of-care” site as “ . . . the place where three elements come together: the patient, the healthcare worker, and care or treatment involving contact with the patient or his/her surroundings.”

The WHO also regards the patient zone as a concept related to the “geographical” visualization of the 5 moments for hand hygiene. It contains the patient and all the surfaces that are touched by or in direct physical contact with the patient, such as examples bed rails, bedside tables, bed linen, infusion tubing or other medical equipment. Surfaces frequently touched by HCWs such as monitor buttons and knobs are also considered part of the patient zone.

The WHO states that there are an estimated 100 million health care workers worldwide, and defines a health care worker (HCW) as anyone whose focus or activity is to improve health. This definition includes providers (e.g., doctors, nurses, clinicians) as well as technicians and managers.

To illustrate some of the problems and difficulties HCWs presently encounter when trying to achieve compliance with WHO's 5 Moments of proper hand hygiene, consider the following scenario as one example:

A HCW enters a patient care zone where upon he/she immediately cleans their hands (Moment 1) using a firmly mounted hand hygiene dispenser, located on a nearby wall, on an equipment stand, or from a bottle type dispenser either carried by the HCW; or, is located on a patient's bed, table, sink counter, etc. The HCW then proceeds to the patient's bed area to begin a patient care service—changing a wound dressing on one of the patient's legs. He/she then lowers the bed railing and removes the patient's bedding from the leg area. In almost all cases, the HCW must now leave the patient and go to a location where a hand hygiene dispenser is located to clean his/her hands (Moments 2 & 5) before performing the changing of the patient's wound dressing. In the meantime, the patient's bed rail has been lowered, which potentially could compromise the safety of the patient, particularly if the patient is under sedation. Upon completing the changing of the wound dressing, the HCW then returns to the location having a hand hygiene dispenser to clean his/her hands (Moments 3 & 4)—again leaving the patient in a potentially unsecure position. The HCW then returns to the patient's bed to raise the bed railing and make other adjustments to the patient's surroundings. The HCW once again returns to a location where a hand hygiene dispenser is located and cleans his/her hands (Moment 5) prior to departing the patient care zone. The HCW has performed a total of four (4) hand hygiene procedures while providing a specific patient care service. Additionally, during the patient care service, the HCW has had to move to and from the patient three (3) times to perform the required hand hygiene procedures.

A survey was conducted in 2007 in healthcare facilities in eastern Ontario, and it was determined that although alcohol based hand rub (ABHR) was available in acute care and long term care facilities, most dispensers are not provided at the point of care. There are numerous research studies that suggest that “bedside” point-of-care hygiene dispensers are most effective in providing HCWs with the opportunity to comply with proper hand hygiene requirements.

Physician adherence to proper hand hygiene within the health care environment remains low. (Hand Hygiene among Physicians: Performance, Beliefs, and Perceptions—Didler Pittet, MD, MS, et al, 2004.)

Results of recently published research conducted at health care facilities, relative to hand hygiene, strongly suggest that the most important place where HCW hand hygiene needs to be performed and monitored is at the patient's “bedside.”

The system described herein provides a cost-effective, unique means in which HCWs can easily perform the required “bedside” hand hygiene procedures, on demand, and at the patient's specific point-of-care location within the patient's care zone and critical sites (patient nest)—such as those described by WHO's “5 Moments Hand Hygiene” protocol. The disclosed system further provides a “networked system” of compliance monitoring of the HCW's “5 Moments Hand Hygiene” performance within all types and demands of patient care zones.

The field of HCW hand hygiene and compliance monitoring therein includes HCW's personal hand hygiene anti-microbial dispensers, such as my previously issued U.S. Pat. Nos. 6,392,546, 7,597,122, 6,883,563, and D512648, and Viillaveces U.S. Pat. No. 5,683,012. Additional art, which relates to fixedly mounted hand hygiene dispensers, includes U.S. Pat. No. 5,945,910 issued to Gorra, and U.S. Pat. No. 6,707,873 issued to Thompson. U.S. Pat. No. 6,727,818 issued to Wildman additionally describes comprehensive methods for monitoring hygiene compliance. Additionally, a May 2009 article in The Journal of the American Osteopathic Association titled “Conspicuous vs Customary Location of Hand Hygiene Agents Dispensers on Alcohol-Based Hand Hygiene Product Usage in an Intensive Care Unit” discloses a novel testing and evaluation technique, which utilizes a movable bed mounted hand hygiene dispenser. GOJO Industries, Inc., additionally discloses within product advertisements, a “patient bed” mounted hand hygiene dispenser that is utilized for patient point-of-care services. Teal patents, LTD discloses within product advertisements, “on-the-move” hand washing devices.

Although existing art describes many methods for dispensing hand hygiene products, and the compliance monitoring of hand cleaning events, they do not provide the means or methods by which a HCW can execute timely, uniform, and monitored hand hygiene dispensing within the patient care zones in point-of-care locations—as recommended by the WHO's “5 Moments Hand Hygiene” protocol.

### SUMMARY

Described briefly, one embodiment has a ceiling mounted transport track, similar to a patient zone privacy curtain track, which is positioned directly above a patient care zone (patient nest) generally concomitant to the perimeter of the patient care zone. The track preferably supports at least one assembly including a flexible and extendable vertically hanging means to support a hand hygiene agents dispenser. The dispenser is easily moved within the transport track, by a HCW, to other locations within the patient nest, to clean his/her hands at the specific location where the HCW will be performing and complying with WHO's 5 Moments Hand Hygiene protocol. When not in use, the hand hygiene dispenser assembly automatically, through the tilted arrangement of the transport track—returns, by gravity, to the docking position located unobtrusively at the “head” area of the patient nest. In this embodiment, the hand hygiene dispenser remains outside a patient's bed area, but also remains generally within the sight, reach, and use of the patient when required.

In yet another embodiment, upon entering a “patient nest” a HCW's unique identifier transceiver badge wirelessly transmits the HCW's unique identifier tag, along with a date/time stamp, to a unique wireless transceiver located in the patient nest. When the HCW enters a patient care zone, the hand hygiene dispenser assembly produces a visual signal and/or sound signal that notify the HCW and the patient that 5 Moments hand hygiene protocols are required. The dispenser's trigger to initiate a visual and/or sound alert can be received from either the “nest” transceiver or the HCW's ID badge. The HCW then moves the hand hygiene dispenser from its docking position to the specific patient care area, within the patient nest, where the HCW will be performing required patient care services. The HCW then, through a touch press pad on the dispenser, enters the WHO's Moment or Moments codes for the procedure(s) the HCW will be immediately providing. The HCW can also, through voice communication, transmit to the transceiver of the dispenser, or the patient's nest transceiver, the Moment or Moments codes. The HCW then causes the hand hygiene dispenser to dispense hand hygiene agents to clean his/her hands, as required by WHO's 5 Moments Hand Hygiene protocol for compliant patient care services.

At the same time the “dispenser” event occurs, the dispenser transmits previously stored HCW event data, to the patient nest's transceiver and concurrently receives a wireless signal from the HCW's unique identifier transceiver, i.e., ID badge, along with a day/date time stamp. The patient nest wireless transceiver can, upon demand, wirelessly transmit each hand hygiene event's time, date, HCW ID #, event location, patient ID, patient procedure, etc. to one of the health care facility's computer memory and processing systems. The HCW's hand hygiene event information is evaluated and measured for compliance to the WHO's hand hygiene “5 Moments” protocol. Wireless transmission of information between the various components of this system is achieved generally by way of: radio frequency; infrared; Wi-Fi; Bluetooth, microwave, voice-to-text, or other wireless technique as will occur to those skilled in the art. In some embodiments, as an example, HCWs' ID badges include active, semi-passive, or passive RFID tags to communicate the identity of the HCW to the system.

A further embodiment provides for multiple hand hygiene dispensers to be utilized within a single patient nest area. Another embodiment provides for a hand hygiene dispenser to be used by two (2) side-by-side patient nests.

According to another embodiment, the hand hygiene dispenser is supported by a flexible and extendable vertically hanging means from a rotating boom assembly, which is mounted to the ceiling directly above the patient nest area. It is preferred that two (2) such booms—one for each side perimeter of the patient nest—be employed.

Another embodiment provides the means for the hand hygiene dispenser to be supported by a moveable, vertically hanging means from an articulated arm assembly that is installed at the vertical wall at the head of the patient care zone, and that is movable above and around the perimeter of the patient nest. Another embodiment provides for two (2) ceiling-mounted, one on each side of the patient bed—in the fore and aft mid-section area of the patient nest—retractable reels that support hanging hand hygiene dispensers which can be extended to the point-of-care patient care service.

Further embodiments include: a means for the hand hygiene dispenser's agents container to be easily refilled or replaced; a continuous supply system for hand hygiene dispenser's agents (for example, drawing from a common reservoir for hand hygiene dispensers in multiple rooms); and the means to provide multiple types of hand hygiene dispensers within a given patient care zone—all collectively enabling full compliance by HCWs with the WHO's 5 Moments Hand Hygiene protocol.

Additionally, a further embodiment provides an audio or visual notification that a specific hand hygiene dispenser needs to be refilled or replaced.

Additional embodiments provide for health care environments, such as an “emergency room,” wherein many HCWs are performing multiple medical care disciplines within the “patient nest.” In such environments, it may be desirous also to have multiple hand hygiene dispensers of various types, e.g., HCW's personal body-worn; wall-mounted; bed-mounted; and/or overhead moveable, in order for HCWs to be able to provide patient care and still maintain compliance with WHO's 5 Moments Hand Hygiene requirements.

Additional embodiments provide for automatic movement of a hand hygiene dispenser to a HCW's location within the patient care zone.

One aspect of the present invention pertains to an apparatus for providing hygiene in a health care facility. Some embodiments include a connector having two ends. Other embodiments include an electronically actuatable dispenser of a hand disinfecting medium attached to an end of the connector. Yet other embodiments include means for suspending the other end of a connector from a surface of the facility; wherein a dispenser is vertically suspended in the patient zone.

Another aspect of the present invention pertains to a method for providing hygiene to a worker in a working environment. Some embodiments include providing a computer and an electronic dispenser of a hand disinfecting medium, the dispenser having a plurality of actuators, the computer being remote from the patient zone and being in electrical communication with the dispenser. Other embodiments include identifying each actuator with a different action chosen from a plurality of actions involving the worker. Yet other embodiments include dispensing an amount of the medium in response to actuating of an actuator by the worker and reporting the identity of each actuating to the remote computer.

Yet another aspect of the present invention pertains to an apparatus for providing hygiene to a worker within a commercial environment. Some embodiments include a source of a hand disinfecting medium. Other embodiments include an electronically actuatable dispenser in fluid communication with a source, the dispenser including a dispensing nozzle. Yet other embodiments include a plurality of marked actuators in electronic communication with a dispenser, the dispenser providing a quantity of the medium from a nozzle in response to actuation of any actuator by the worker; wherein each actuator is marked differently than each other actuator and each marking pertains to a different action by the worker chosen from a plurality of predetermined actions by the worker within the zone.

Still another aspect of the present invention pertains to a method for providing hygiene to worker in a commercial facility. Some embodiments include providing a computer and an electronic dispenser of a cleaning medium, the dispenser having a plurality of actuators, each actuator having an annunciator, the computer being in electrical communication with the dispenser. Other embodiments include programming the computer with a list of predetermined actions, each action involving both the commercial facility and the worker. Yet other embodiments include identifying each actuator with a different action chosen from the plurality of predetermined actions. Still other embodiments include triggering the annunciator of at least one actuator by the computer, and turning off the one annunciator in response to actuating the annunciated actuator.

Yet another aspect of the present invention pertains to a method for providing hygiene to a worker in a commercial facility. Some embodiments include providing an electronic recording device and a dispenser of a cleaning medium, the dispenser having at least one actuator, the dispenser having a display, the recording device being in electrical communication with the dispenser. Other embodiments include displaying with the display a plurality of predetermined actions, each action involving both the facility and the worker. Still further embodiments pertain to actuating the actuator to dispense the medium, and preparing a data record of said actuating.

Still another aspect of the present invention pertains to an apparatus for providing hygiene to a health care worker within a patient zone of a health care facility. Some embodiments include means for dispensing a cleaning medium, means for suspending the dispensing means, means for identifying the dispensing means, and means for recording compliance of the usage of the dispensing medium. Each of the dispensing means, suspending means, identifying means, and compliance recording means can be expressed in a variety of different ways, as found herein in the claims, specification, and drawings, and also as known to those of ordinary skill in the art. It is understood that each of the variants of these four means can be combined with any variant of any of the other means. It is beyond reason to express each and every one of the many possible combinations of variants, although these variations will be discernible to those of ordinary skill in the art.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

FIG. 1 is a drawing that represents, in part, a patient care zone or “patient nest” and the WHO's 5 Moments Hand Hygiene protocol.

FIG. 2 is a photographic representation of the patient care zone “nest” showing one or more embodiments of the present invention.

FIG. 3 is another representation of the patient care zone “nest” showing one or more embodiments of the present invention.

FIG. 4 is another representation of the patient care zone “nest” showing one or more embodiments of the present invention.

FIGS. 5A, 5B, and 5C are drawings showing an embodiment of the hand hygiene assembly movement within the patient nest.

FIGS. 6A, 6B, and 6C are drawings showing one or more embodiments of the present invention relating to the hand hygiene dispenser assembly.

FIGS. 7A, 7B, and 7C are drawings showing alternative embodiments relating to the hand hygiene dispenser assembly.

FIGS. 8A, 8B, and 8C are drawings showing an embodiment of the hand hygiene assembly's movement within the patient nest.

FIGS. 9A, 9B, and 9C are drawings showing an alternative embodiment of the hand hygiene assembly's movement within the patient nest.

FIGS. 10A, 10B, and 10C are drawings showing another alternative embodiment of the hand hygiene assembly's movement within the patient nest.

FIG. 11 is a block diagram drawing showing embodiments of the system information transmission network.

FIG. 12 is a photographic view of another embodiment showing a patient's “bed mounted” hand hygiene dispenser incorporating a WHO's 5 Moments Hand Hygiene input means.

FIG. 13 is a photographic view of another embodiment showing a patient nest's hand hygiene dispenser incorporating a WHO's 5 Moments Hand Hygiene input means.

FIG. 14 is a photographic view of another embodiment showing a hand hygiene dispenser incorporating a WHO's 5 Moments Hand Hygiene input means—being worn by a HCW.

FIG. 15 is a perspective view showing an embodiment of a remotely mounted hand hygiene agents supply container with pumping means and a mechanized transport apparatus for a hand hygiene dispenser.

FIG. 16 is a perspective view showing further detail of a mechanized transport apparatus for a hand hygiene dispenser.

### DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention. It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “preferably,” refers to features and aspects that are present in at least one embodiment, but which are optional for some embodiments.

The use of an N-series prefix for an element number (NXX.XX) refers to an element that is the same as the non-prefixed element (XX.XX), except as shown and described thereafter. As an example, an element 1020.1 would be the same as element 20.1, except for those different features of element 1020.1 shown and described. Further, common elements and common features of related elements are drawn in the same manner in different figures, and/or use the same symbology in different figures. As such, it is not necessary to describe the features of 1020.1 and 20.1 that are the same, since these common features are apparent to a person of ordinary skill in the related field of technology. This description convention also applies to the use of prime (′), double prime (″), and triple prime (′″) suffixed element numbers. Therefore, it is not necessary to describe the features of 20.1, 20.1′, 20.1″, and 20.1′″ that are the same, since these common features are apparent to persons of ordinary skill in the related field of technology.

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

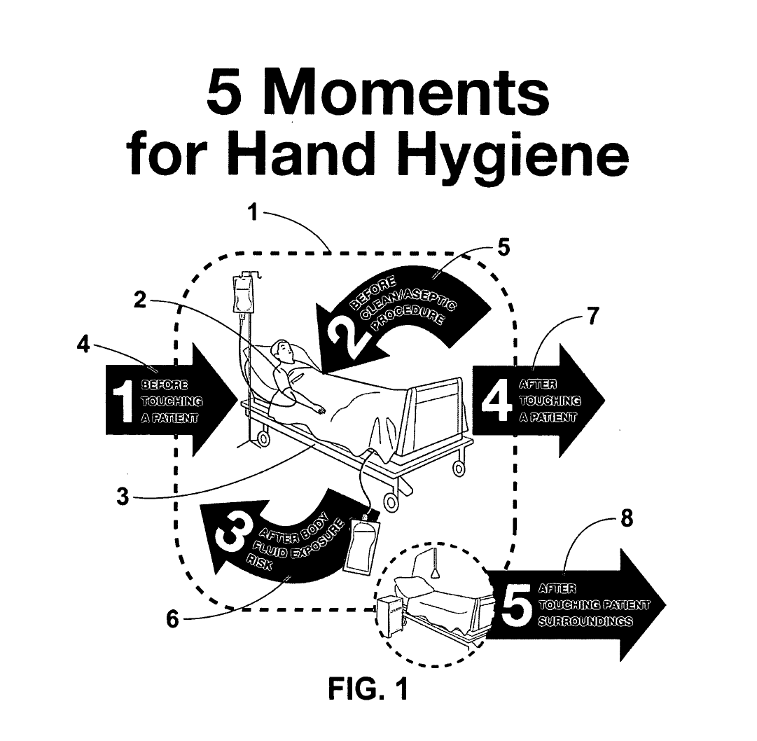

Referring now to FIG. 1. The drawing represents, in part, a patient care zone or patient nest (1); a patient (2); a patient's bed (3); and the WHO's Five Moments for Hand Hygiene—Moment 1 (4); Moment 2 (5); Moment 3 (6); Moment 4 (7); and Moment 5 (8).

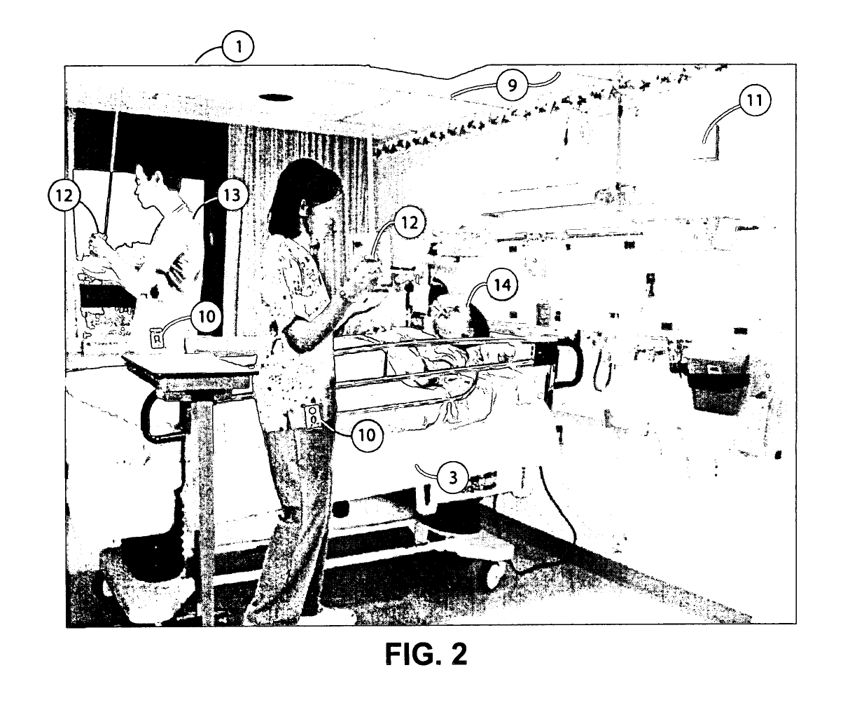

FIG. 2 is a pictorial representation showing healthcare workers (HCWs) (13) performing one of the WHO's 5 Moments Hand Hygiene events (4, 5, 6, 7, 8) within the patient nest (1). Upon entering the patient nest (1) HCW(s) (13) ID transceiver badge (10) wirelessly transmits a unique signal to the patient nest transceiver (11) and to the hand hygiene dispenser's (12) transceiver. The hand hygiene dispenser is suspended and supported by a ceiling (19)-mounted perimeter transport track (9). When the patient nest's transceiver (11) receives the HCW's ID transceiver badge's unique wireless transmission, it immediately wirelessly transmits a unique wireless signal to the hand hygiene dispenser's transceiver.

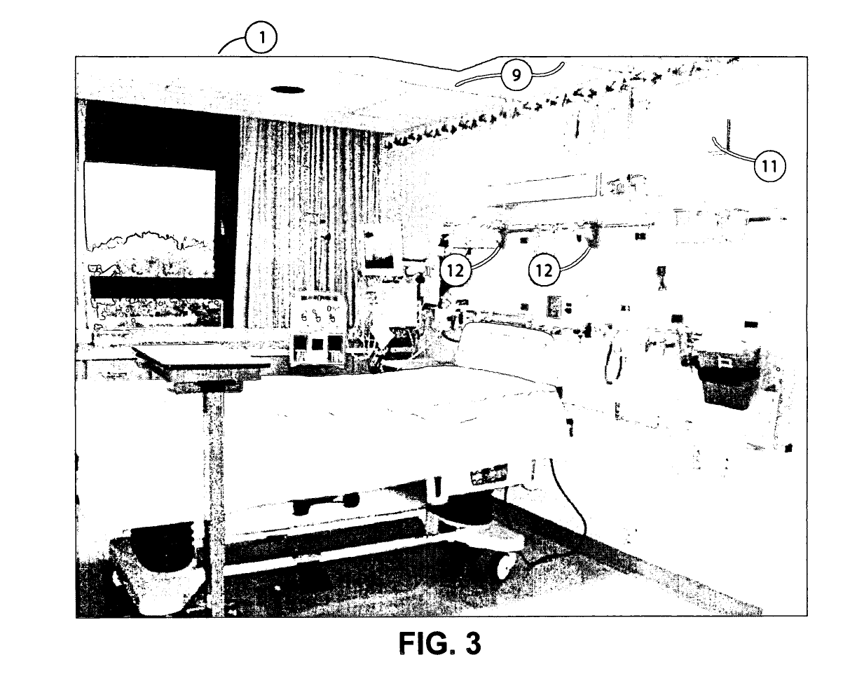

FIG. 3 is another pictorial representation showing an unoccupied patient nest (1) with the hand hygiene dispenser in a “not in use” state located at the “docking” position at the fore section of the patient's bed (3) area.

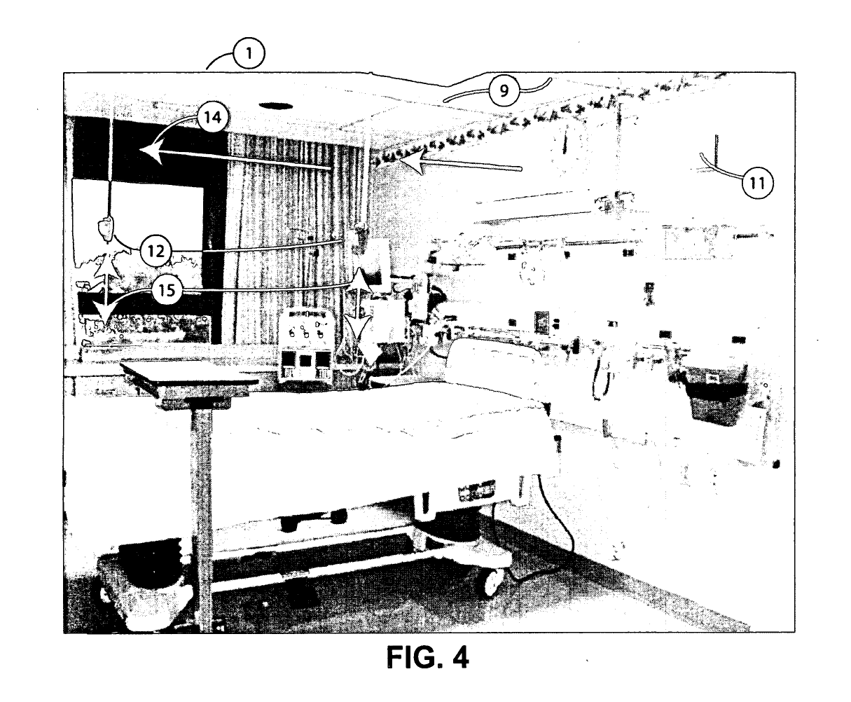

FIG. 4 is yet another pictorial representation showing an un-occupied patient nest (1) wherein the hand hygiene dispensers are shown in movement fore and aft (14) and showing up-and-down movement (15).

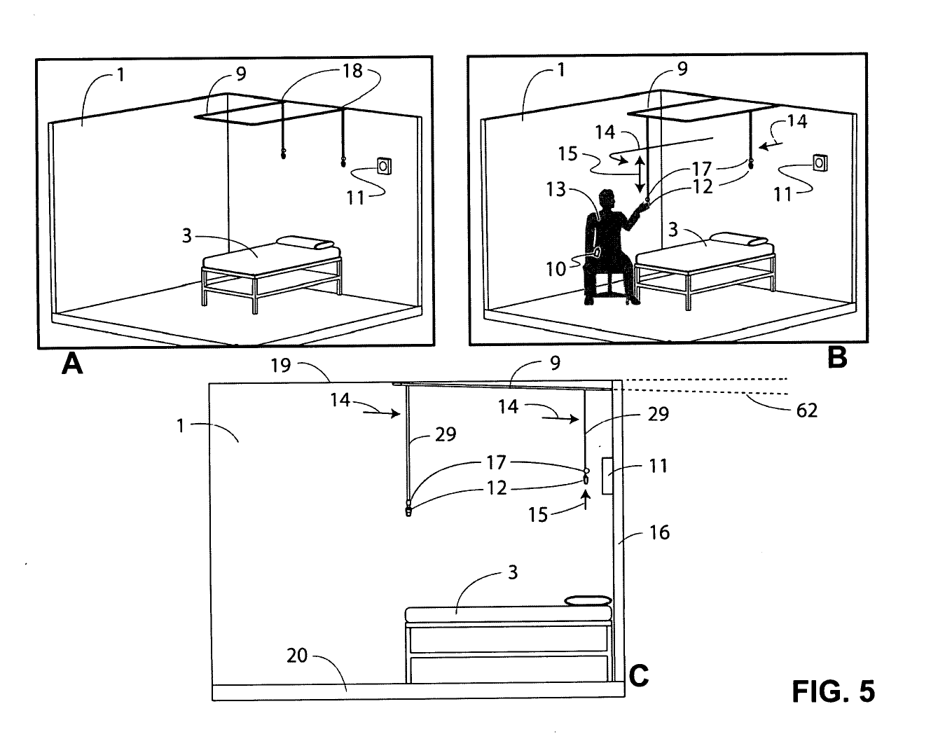

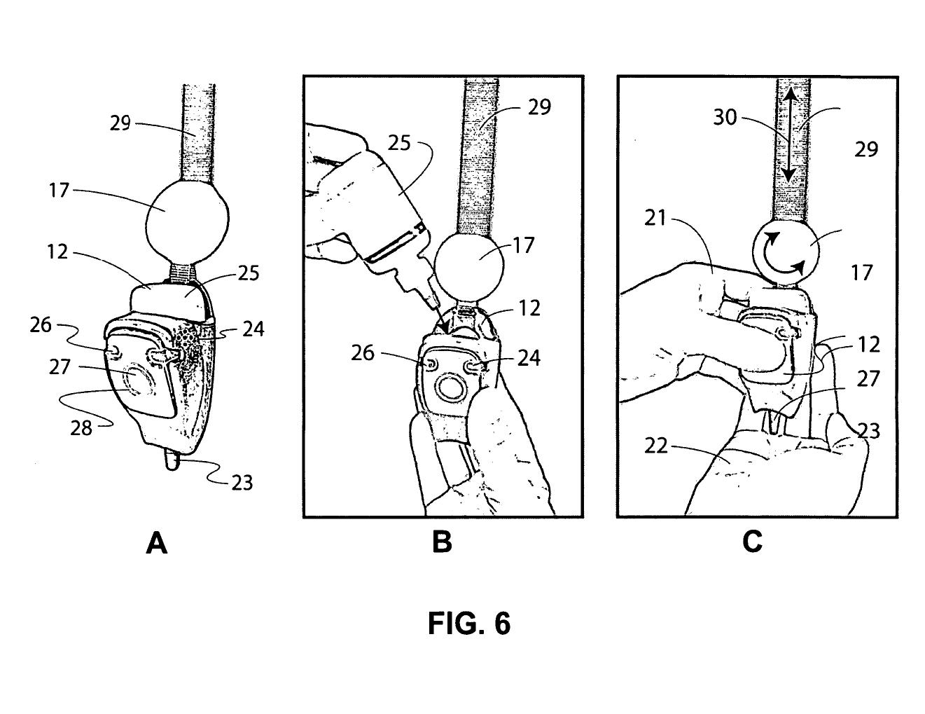

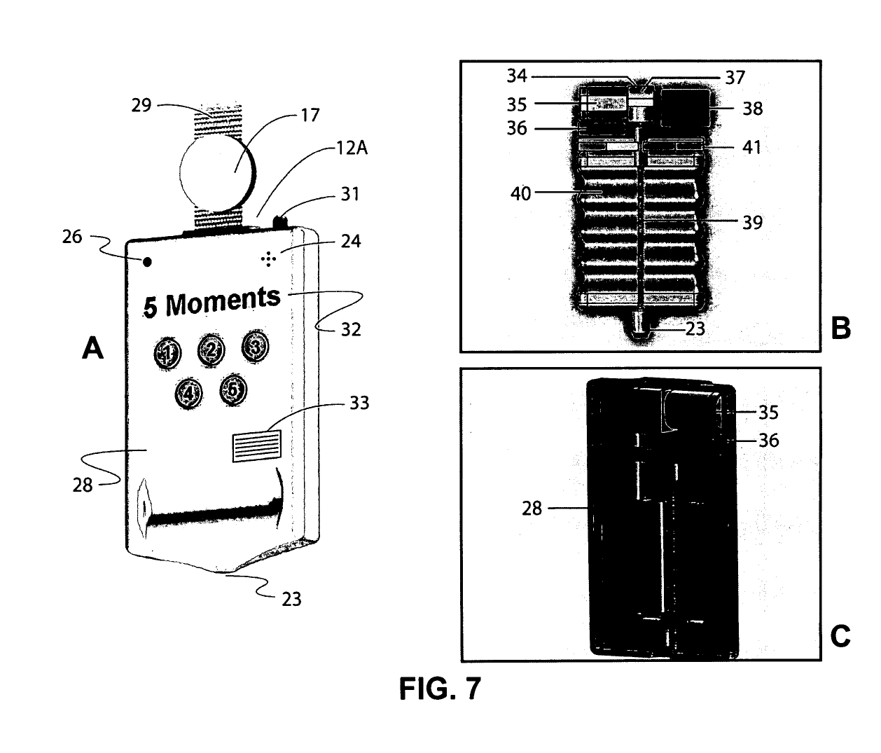

Referring now to the embodiment as shown in FIGS. 5A, 5B, 5C, 6A, 6B, 6C, and 7A, 7B, and FIG. 7C:

The patient care zone or patient nest (1) has a ceiling (19) mounted transport track (9) that is generally positioned around the perimeter of the patient's bed (3). The patient nest has a fore-positioned wall (16) located at the head of the patient bed (3). The transport track (9) has a “not-in-use” docking position (18) at the general location of the vertical wall (16). Although what has been shown and described is a patient care zone (1) in which the docking position is shown near the head position of bed (3), it is understood that the docking position can be anywhere within the nest (1). In addition, although what has been shown and described pertains in one instance to a patient care zone within a health care facility, it is understood that yet other embodiments of the present invention pertain to a worker in any working, commercial environment in which the hygiene of the worker is important. An example of such a commercial facility would be a restaurant.

When a HCW (13) enters the patient nest (1), his/her ID badge (10) transceiver wirelessly transmits a unique signal to the patient nest's transceiver (11). The patient nest (1) transceiver (11) sends a wireless signal to the hand hygiene dispenser's transceiver (28), which causes the dispenser (12) to emit an alert, for example, a light (26) and/or audio tone (24). The HCW (13) moves the hand hygiene dispenser (12) from its “docking” location (18) along to a position (14) where the HCW (13) will be performing a patient care service requiring at least one of WHO's 5 Moments (FIG. 1) Hand Hygiene events. There are several manufacturers who provide economical ceiling-mounted transport tracks (9) such as Medline's Cubicle DRL-100 model. Once the hand hygiene dispenser (12) is moved (14) laterally to the use position, the HCW (13) can then position the hand hygiene dispenser (12) to a preferred lowered position (15) for precise hand hygiene dispensing. By way of example, a HCW (13) is shown in a seated position (13B).

The HCW (13) then, for example, inputs the WHO's Moment code (4) (FIG. 1) into the hand hygiene dispenser (12A) press pad or touch screen (32). The HCW (13) can also issue an audio voice command (33) to enter the code.

Once the hand hygiene dispenser (12A) has had an input from a HCW (13), the dispenser's (12,12A) light (26) and/or audio (24) alert is turned off. If the hand hygiene dispenser (12) does not have a 5 Moments Hand Hygiene event input means, then the dispenser's (12,12A) alert light (26) and/or audio (24) is cancelled by the HCW's (13) action to cause the dispenser's (12, 12A) to dispense hand hygiene agents through dispenser (12) press pad (27) or dispenser (12A) motion sensor (31).

As previously described, HCW (13) moves the dispenser (12, 12A) fore and aft (14) and side-to-side (14) (FIG. 5) by means of transport track (9) and moves the dispenser (12, 12A) in a up-and-down (29B) (29A) movement (15) by means of a spring-loaded recoiler (17). The recoiler (17) is shown directly attached at the aft end of the dispenser (12, 12A), although the recoiler (17) can be attached anywhere between the transport track (9) and the dispenser (12, 12A).

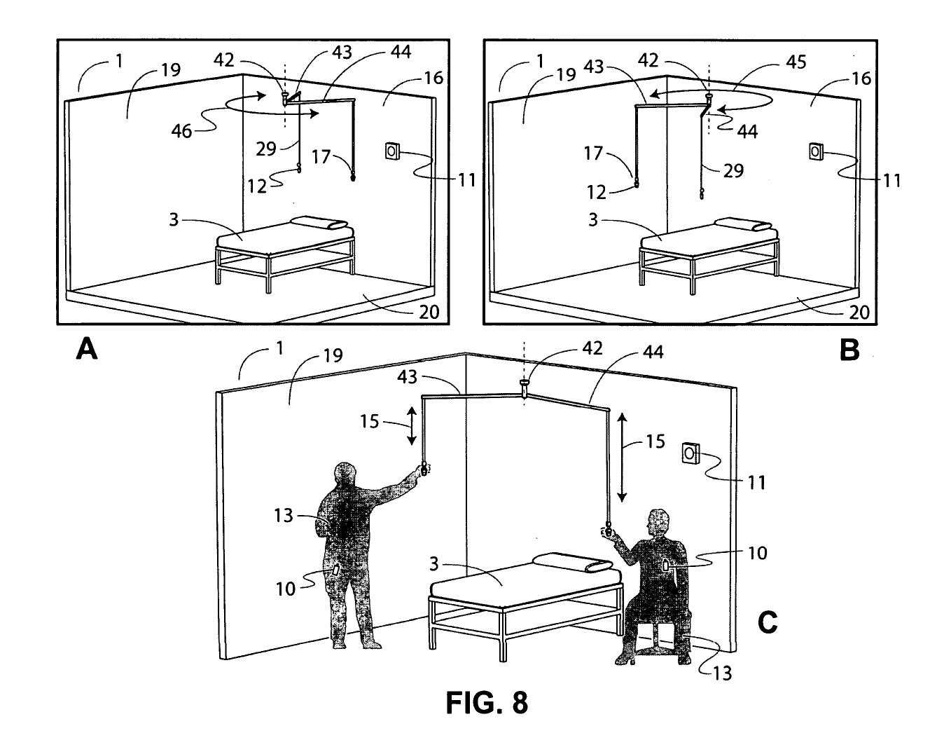

Now referring to another embodiment (FIG. 8) that relates to a movable hand hygiene dispenser within a patient care zone which can be quickly installed in a mobile or portable health care facility, e.g., military MASH Unit (Mobile Army Surgical Hospital). Ceiling (19)-mounted swing arms (43) and (44) are pivotably attached to a swivel joint (42), which is generally located above the fore-and-aft and side-to-side centerlines of the patient's bed (3), and which move and operate around the perimeter of the patient nest. Each swing arm (43)(44) has a vertically extending flexible tether (29) that is connected to a recoiler (17) and hand hygiene dispenser (12). When not-in-use, each swing arm has a docking position of (46) located generally next to the patient nest's vertical wall (16) at the patient's bed (3) area. Swivel joint (42) has a slight tilt (or coiled spring bias) from a vertical alignment, which provides a tendency for a gradual return (46) of each swing arm (43)(44), when not in use, to naturally return through gravity (or coiled spring) to the docking position (44). There are several manufacturers that make ceiling-mounted swing arm joints with attaching swivel arms. One such manufacturer is the Zierden Company that offers the Zierco Model Twin 360 Degrees that, in part, represents part of the apparatus described and shown in FIG. 8.

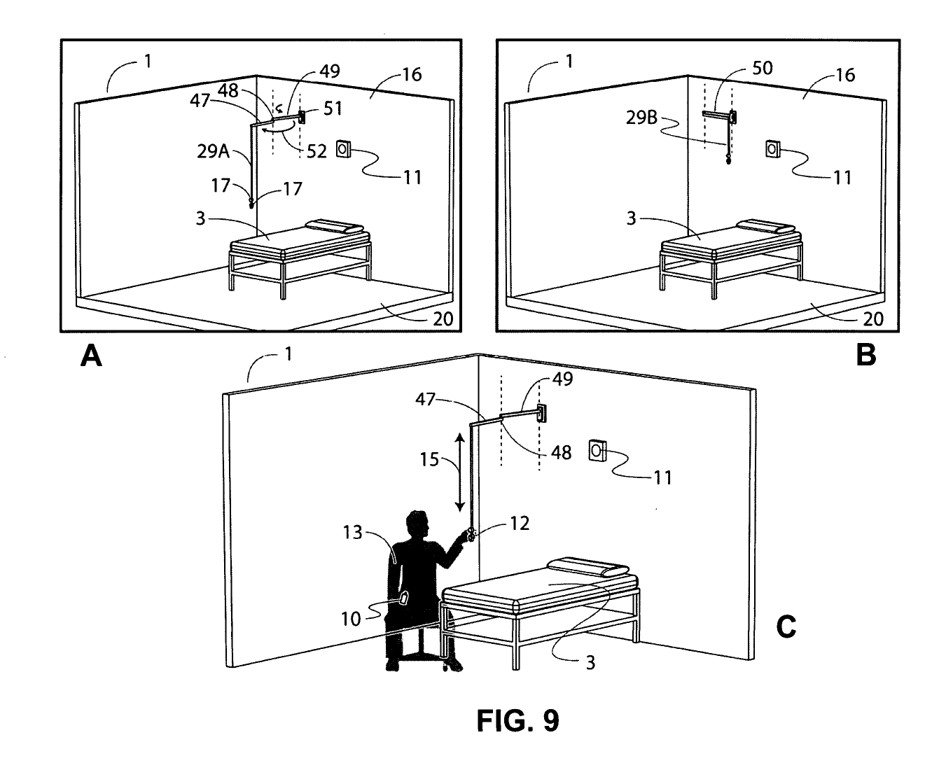

An alternative embodiment for a mobile moveable patient nest hand hygiene dispenser, which can be quickly installed in a MASH type health care facility that does not have a structural support ceiling, would include a spring-biased, articulated swing arm which is mounted directly to the vertical wall in the patient nest area at the head of the patient bed area as shown in FIG. 9. The vertical wall (16) supports pivot mounting means (51), which in turn supports a primary arm (49) and a secondary arm (47). The primary arm (49) is connected to the secondary arm (47) through a connecting joint (48). The docking position—for when the dispenser is not in use—is shown at (50). The rotational direction of the articulated arm movement is shown at (52). The dispenser (12) having recoiler (17) is connected to the secondary arm (47) by means of a flexible tether, shown alternatively; retracted (29B) and in the extended position (29A). One (1) articulated arm arrangement is shown in FIG. 9—though embodiments using two (2) articulated arm assemblies are able to move around the perimeter of the patient nest. Manufactured articulated arm assemblies, such as Sanus System's product Model MF215-B1 “Full Motion Mount” is a representation, in part, of a type of vertical wall-mounted articulated arm overhead support means.

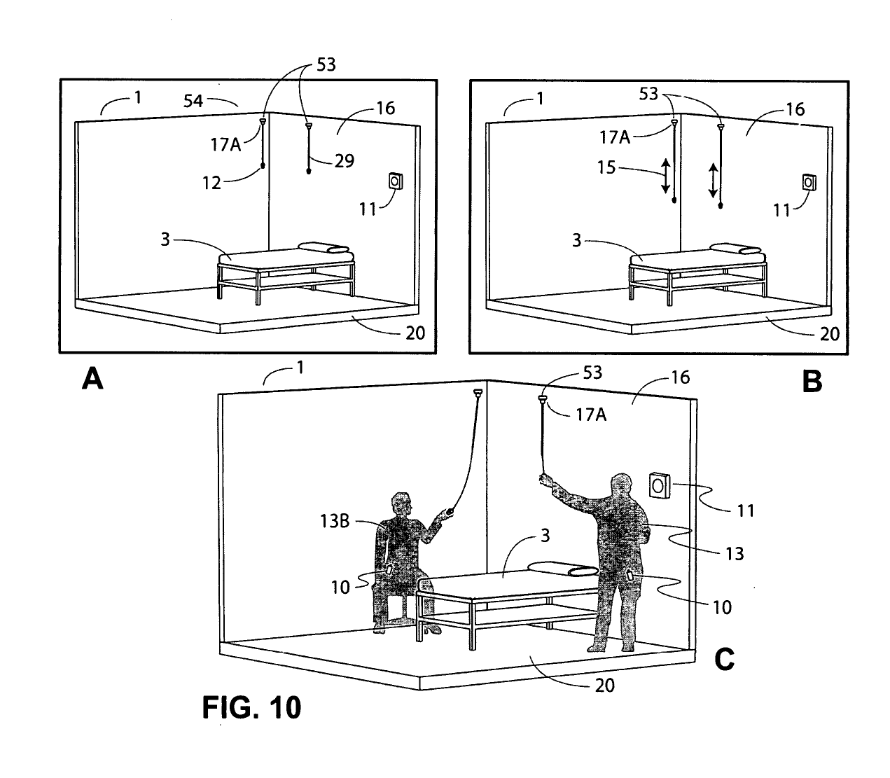

Another embodiment is a ceiling mounted (patient bed area) (54) and supported hand hygiene dispenser assembly which relates to “compact” health care facilities that are located within a mobile medical transport trailers such as what is shown in FIGS. 10A, 10B, and 10C.

In this embodiment, the hand hygiene dispenser (12) is directly suspended at the mid-centerline perimeter of a patient's bed, by means of a flexible tether (29A, 29B) from a ceiling (54) mount (53) directly connected to a recoiler (17A). Ametek Inc. manufactures a product line of recoilers/reels such as D220911-2 Sureshield.

There are many companies that manufacture recoilers, such as: The RTF Company's 02 Series, or Oceanic Company's VT4 Retractor Assembly, which represent, in part, the type of overhead and/or remote mounted dispenser recoilers that would support a hand hygiene dispenser assembly as described and shown in FIG. 5 through FIG. 10.

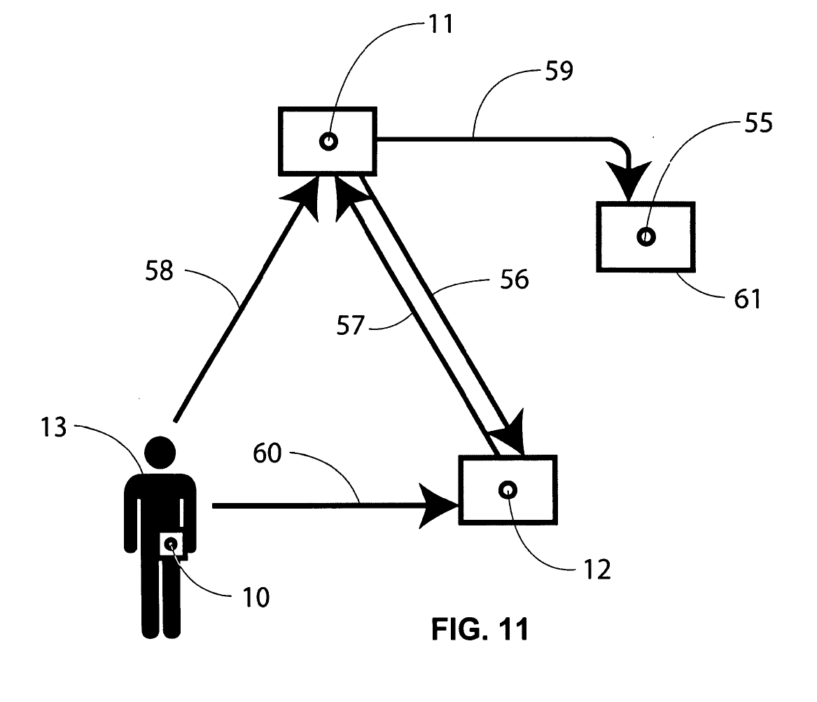

What has been previously shown and described herein represents various embodiments for different types of patient care zone “patient nests” and the hand hygiene systems that are uniquely utilized by HCWs to be able to fully comply with the WHO's 5 Moments Hand Hygiene protocols within the patient nests. Now referring to FIG. 11, which describes and shows how some embodiments of the hand hygiene systems operate, and how the HCW's use compliance is monitored when using the dispenser system to perform the WHO's 5 Moments Hand Hygiene events: The patient nest has a transceiver (11) capable of wirelessly transmitting or receiving data. The HCW (13) has a unique ID badge (10) that is a transceiver that is capable of wirelessly transmitting and receiving data; a hand hygiene dispenser (12) that is also capable of wirelessly transmitting and receiving data. The HCW's ID badge transceiver wirelessly sends data (58) to the patient nest transceiver (11) and (60) to a hand hygiene dispenser (12). The hand hygiene dispenser (12) wirelessly transmits data (57) to the patient nest transceiver (11) and wirelessly receives data from the patient nest transceiver (11). The patient nest transceiver (11) also wirelessly transmits data (59) to the health care facility's computer processing system (55). The health care facility's computer processing system processes the data that it wirelessly receives (59) from the patient nest transceiver (11) and can publish WHO 5 Moments Hand Hygiene Compliance Reports (61).

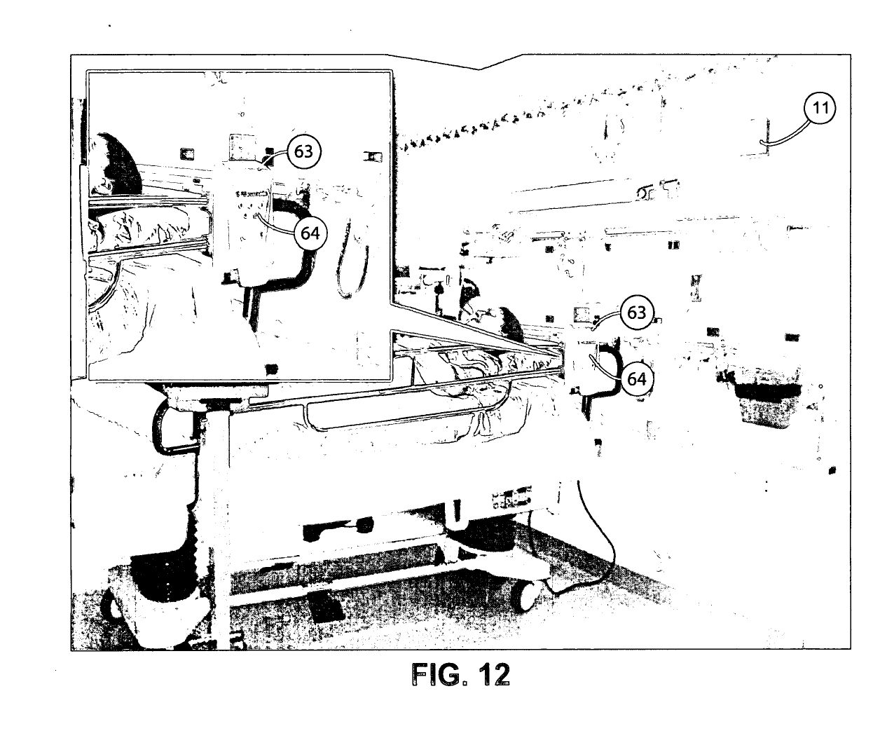

In some health care environments, such as an emergency room, wherein many HCWs are performing multiple medical care disciplines within the “patient nest,” it may be desirous also to have multiple hand hygiene dispensers of various types, e.g., HCW's personal body-worn; wall-mounted; bed-mounted; and overhead moveable, in order for HCWs to be able to provide speedy patient care and still maintain compliance with WHO's 5 Moments Hand Hygiene requirements. FIG. 12 shows a pictorial view of a patient nest (1); a patient (2); a patient bed (3); a patient bed-mounted hand hygiene dispenser (63) having an input means to receive WHO's 5 Moments Hand Hygiene events (64); and a patient nest transceiver (11).

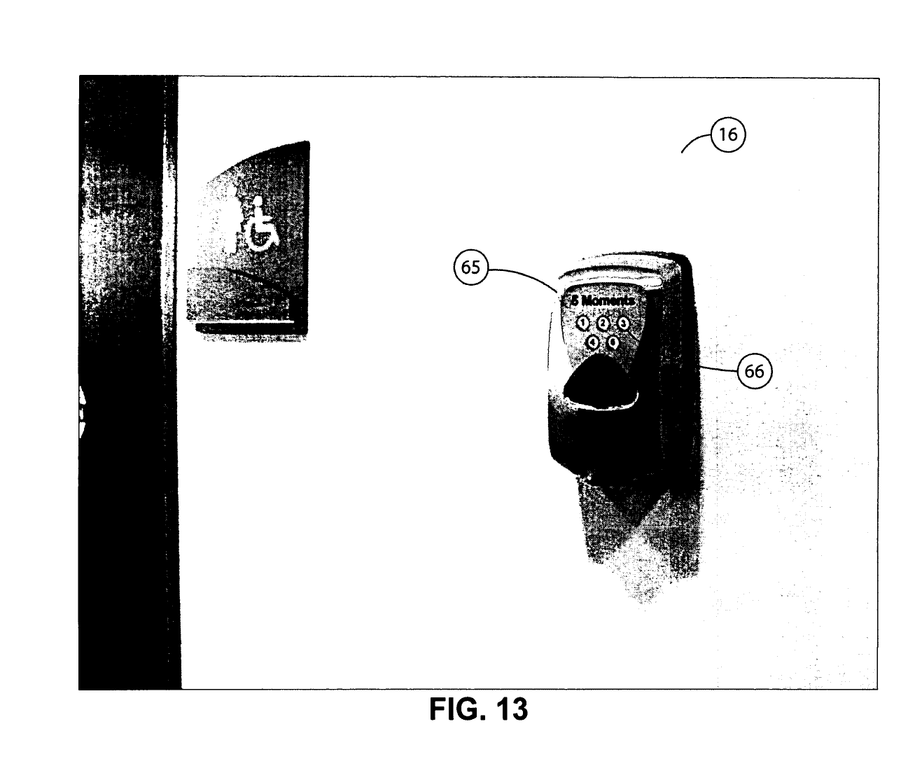

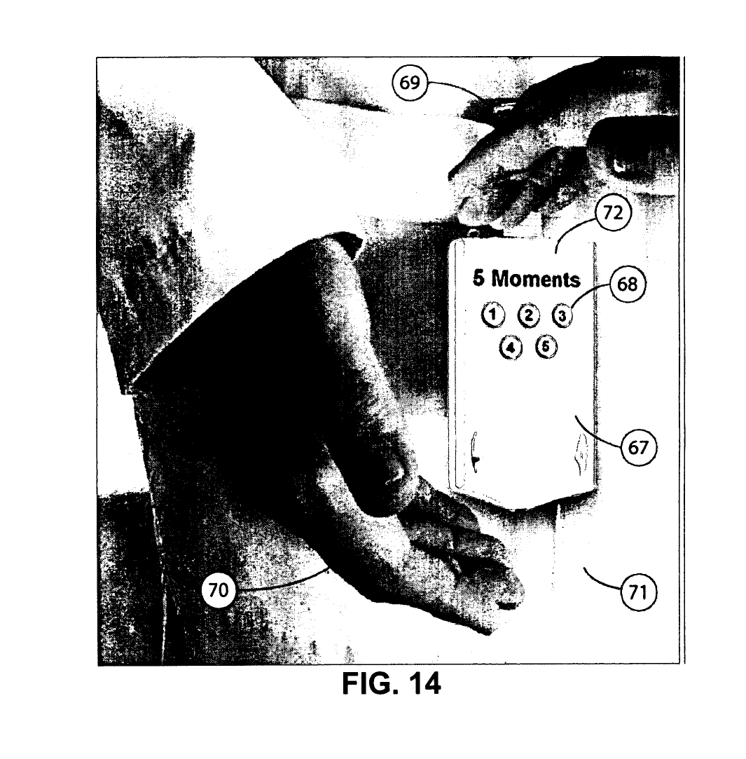

FIG. 13 shows a pictorial view of a patient nest's vertical wall (16) having a wall-mounted hand hygiene dispenser (65) having an input means to receive WHO's 5 Moments Hand Hygiene events (66) and a patient nest transceiver (which is not shown in the pictorial view). FIG. 14 shows a HCW (13) wearing a infrared-activated hand hygiene dispenser (67) attached (69) to the HCW's coat (71). The HCW's (13) right hand (70) is shown receiving hand hygiene agents from the HCW's left hand's (72) action of passing over the motion sensor (31), causing agents to be dispensed from the dispenser. The hand hygiene dispenser (67) has an input means to receive a WHO's 5 Moments Hand Hygiene events (68) and a patient nest transceiver (which is not shown in the pictorial view). The above-described embodiments of the bed-mounted hand hygiene dispenser (63); the wall-mounted dispenser (65); and the HCW personal, body-worn dispenser (67) all have the means to transmit and receive data wirelessly, as has been previously described herein.

Various embodiments of the present invention incorporate a close functional relationship between the matter that is printed on the actuator (66) of the dispenser and the reasons for the healthcare worker to use that particular actuator. The printed indicia are not merely an aesthetic choice of design. There is a functional relationship between the printed indicia and the function of the actuator that is preferably established by the operators of the healthcare facility. Therefore, the indicia denotes a specific function, as well as a functional relationship with the underlying substrate, in that it enables the healthcare worker to associate a dispensing actuation with a particular act of patient care, and further in those embodiments including computer, to create a business record establishing that a proper hygienic protocol was followed.

Referring to FIG. 13, it can be seen that in some embodiments the dispenser includes as actuators a plurality of buttons, and each button is associated with a number, and those numbers are associated with the concept of the 5 Moments protocol.

However, yet other embodiments of the present invention contemplate other types of indicia printed on the actuator that represent 1 of the 5 Moments protocol. As another example, instead of the numerals 1-5, each actuator could include a pictorial representation of the particular Moment (as one example, a bed and an arrow pointing to the bed representing Moment 1, or a pictorial representation of a catheter inserted in an arm of a patient to represent Moment 3, and the like). Still further, each of the actuators could include printed indicia that includes a selection of words representing the particular Moment (as examples, the words “before touching” to represent Moment 1, the words “before procedure” to represent Moment 2, and the like).

Further, it is understood that the actuator of the dispenser can be of any type, including buttons for touch actuation by a healthcare worker, a touch screen for touch actuation by a healthcare worker (with the printed indicia being shown on the print screen), as examples. Further, in those embodiments in which the dispenser is voice actuated, the actuation command is representative of the particular Moment (as examples, the statements “body fluid” or “Moment 3” to represent a dispensing actuation for Moment 3). Further, it is understood that the printed indicia can also be sensed in a tactile manner (such as an embossed “1” to represent Moment 1, or four embossed geometric shapes such as circular projections to represent Moment 4).

Further, although what has been shown and described are actuators that have printed indicia functionally related to a purpose of the actuator related to one of the 5 Moments, it is further understood that the various actuators and printed indicia are not limited to the 5 Moments, and can represent any type of hygienic operation within the patient zone. Preferably, even though each of the plurality of actuators is represented with a unique printed indicia corresponding to a unique hygienic moment, the functional purpose of the actuators are substantially identical, with the actuation of each actuator resulting in a dispensing of a quantity of a disinfectant medium.

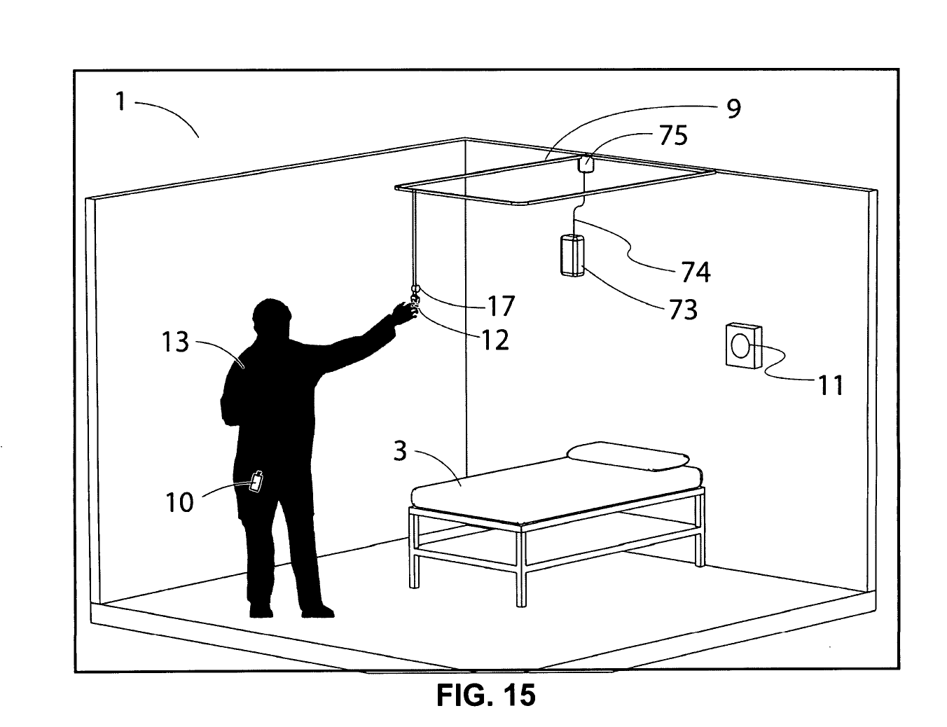

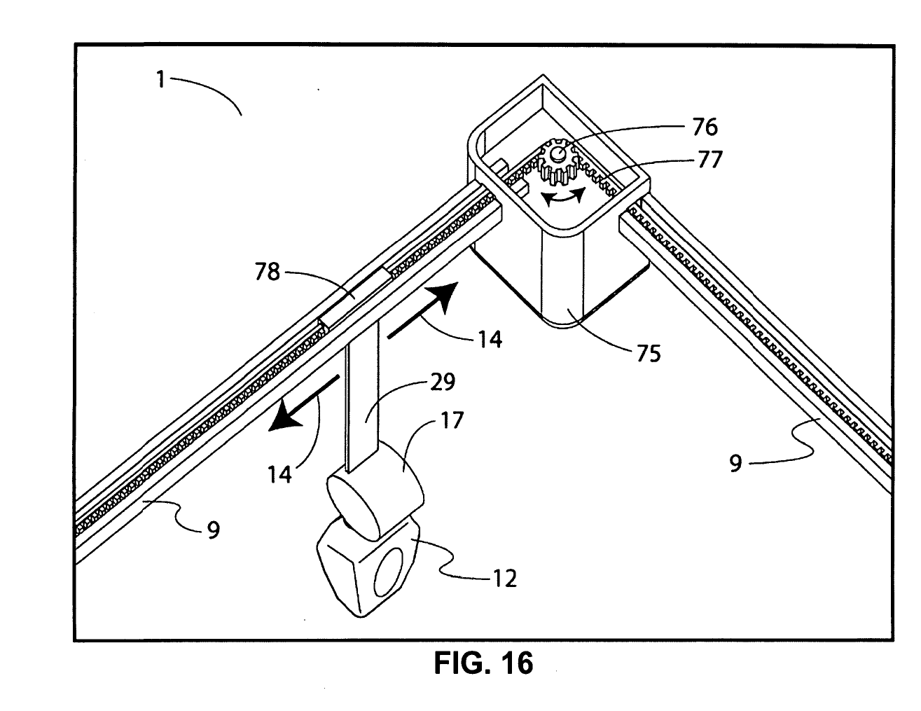

In certain health care facilities having patient care zones dedicated to performing and providing high-frequency, specific-patient care services, it may be desirous to have hand hygiene dispensers that have agents containers that are automatically supplied with agents from a remotely mounted supply source. Additionally, it may be advantageous to have hand hygiene dispensers moved automatically to the location within the patient's care zone, at the point of care, wherein the HCW performs a “5 Moments” hand hygiene event. In such patient care environments, as shown in FIG. 15 and FIG. 16, agents supply source (73) delivers hand hygiene agents through agents discharge line (74) to dispenser assembly (12). The agent's delivery system, e.g., pumps, gravity feed, etc., can be provided by any of numerous manufacturers within the respective industry.

As previously described, another embodiment provides for the automatic transport of a hand hygiene dispenser within the transport track to the point-of-care at which a HCW will be performing a “5 Moments” hand hygiene event. When a HCW (13) enters the patient care zone (1) his/her transceiver ID badge (10) transmits a proximity wireless signal to activate a proximity controller and drive motor (75), which rotates drive gear (76) to linearly move drive belt (77) in a fore and aft lateral motion within the ceiling mounted transport track (9) to an automatic “stop” location proximate to where the HCW (13) is positioned. (As previously described, automatic movement of the “dispenser” could be activated by a HCW's voice command.) The hand hygiene dispenser assembly (12) is connected to the drive belt (77) by connecting link (78). When the HCW (13) exits the patient care zone (1) the hand hygiene dispenser assembly (12) automatically returns to its docking location (18).

Various aspects of some embodiments of the present inventions include the following:

Methods/means for HCWs to perform and register WHO's 5 Moments Hand Hygiene events within a patient care zone.

Moveable hand hygiene dispenser within a patient care zone.

Moveable and fixedly mounted hand hygiene dispensers within a patient care zone.

Monitoring a HCW's WHO's 5 Moments Hand Hygiene dispenser events within a patient care zone.

In a health care facility, within the immediate patient care zone, a method of having at least one (1) hand hygiene dispenser, flexibly mounted within the patient zone, and having means for at least one (1) HCW to perform at least one (1) Moment of WHO's 5 Moments Hand Hygiene events.

In a health care facility, within the immediate patient care zone, a method of receiving and transmitting information relative to at least one (1) of the HCW's performing at least one (1) of WHO's 5 Moments Hand Hygiene events.

In a health care facility, within the immediate patient care zone, having at least one (1) HCW, at least one (1) moveable hand hygiene dispenser, at least one (1) fixedly mounted hand hygiene dispenser, and a means to receive and transmit information relative to at least one (1) HCW performing at least one (1) of WHO's 5 Moments Hand Hygiene events performed using the moveable and or fixedly mounted hand hygiene dispenser(s).

In a health care facility, the method of collecting, evaluating, and reporting a HCW's hand hygiene event(s) within a patient care zone(s), relative to the WHO's 5 Moments Hand Hygiene protocol.

In a health care facility, within the immediate patient care zone, a method for a HCW to perform at least one (1) of the following WHO's Hand Hygiene events, and the means to record, evaluate, and report the HCW's WHO's Hand Hygiene events; before touching the patient, before clean/aseptic procedures for the patient, after body fluid exposure/risk from the patient, after touching the patient, and after touching the patient's surroundings.

The method of any of the preceding statements, wherein a protractible hand hygiene dispenser is movable within the patient nest by the HCW.

The method of any of the preceding statements, wherein the hand hygiene dispenser is flexibly connected to a movable means to provide transport of the dispenser within the patient nest.

The method of any of the preceding statements, wherein the hand hygiene dispenser is fixedly connected to a movable means to provide transport of the dispenser within the patient nest.

The method of any of the preceding statements, wherein a hand hygiene dispenser is movable within the patient nest area by a HCW.

The method of any of the preceding statements, wherein the hand hygiene dispenser is suspended in a plane above the patient nest area.

The method of any of the preceding statements, wherein the hand hygiene dispenser is in a flexibly hanging type vertical position with the dispenser's fluid outlet port facing the floor of the patient nest area.

The method of any of the preceding statements, wherein two (2) or more HCWs can utilize hand hygiene dispensers at the same time within the same patient nest area.

The method of any of the preceding statements, wherein the hand hygiene dispenser movement within the patient nest is executed by a ceiling-mounted transport track.

The method of any of the preceding statements, wherein the hand hygiene dispenser movement within the patient nest is executed by a ceiling-mounted rotatable suspension arm.

The method of any of the preceding statements, wherein the hand hygiene dispenser movement within the patient nest is executed by a one or more wall-mounted articulating pivot arms.

The method of any of the preceding statements, wherein the hand hygiene dispenser movement within the patient nest is executed by a ceiling-mounted recoiler.

The method of any of the preceding statements, wherein the hand hygiene dispenser has means to provide audio and/or visual alerts within the patient nest.

The method of any of the preceding statements, wherein the hand hygiene agent's container is replaceable.

The method of any of the preceding statements, wherein the hand hygiene agent's container is refillable.

The method of any of the preceding statements, wherein the hand hygiene agent's container is continuously filled from a remote pump and agents supply reservoir.

The method of any of the preceding statements, wherein the hand hygiene dispenser has a unique identifier which is wirelessly connected to a data collector transceiver which records a HCW's unique identifier, location, date, time of a hand hygiene event.

The method of any of the preceding statements, wherein the hand hygiene dispenser wirelessly transmits a HCW's unique identifier, hand hygiene unique identifier, location, date, time of a hand hygiene event to a data collector.

The method of any of the preceding statements, wherein the hand hygiene dispenser wirelessly receives a HCW's unique identifier and retains if in a memory.

The method of any of the preceding statements, wherein the HCW's ID badge is the data collector and the hand hygiene dispenser is the transmitter.

The method of any of the preceding statements, wherein the patient nest has a unique identifier.

The method of any of the preceding statements, wherein the hand hygiene dispenser dispenses hand hygiene agents for the HCW's use, without the need for the HCW to physically touch the dispenser.

The method of any of the preceding statements, wherein the dispenser assembly visually displays a flashing light when a HCW enters a patient care zone.

The method of any of the preceding statements, wherein the dispenser assembly audibly annunciates when a HCW enters a patient care zone.

The method of any of the preceding statements, wherein a movable hand hygiene dispenser and a fixedly mounted hand hygiene dispenser are simultaneously used by a plurality of HCWs within a patient care zone.

The method of any of the preceding statements, wherein a personal hand hygiene dispenser and a fixedly mounted hand hygiene dispenser are simultaneously used by HCWs within a patient care zone.

The method of any of the preceding statements, wherein a movable hand hygiene dispenser; a fixedly mounted hand hygiene dispenser; and a personal hand hygiene dispenser are simultaneously used by HCWs within a patient care zone.

The method of any of the preceding statements, wherein a HCW inputs through a hand hygiene dispenser data recording the occurrence of at least one (1) of the WHO's 5 Moments Hand Hygiene events.

The method of any of the preceding statements, wherein the HCW's transmission is voice command.

The method of any of the preceding statements, wherein the HCW's transmission is through a touch screen.

The method of any of the preceding statements, wherein the HCW's transmission is through a press pad.

The method of any of the preceding statements, wherein multiple HCWs utilizing multiple hand hygiene dispensers can substantially simultaneously, within a single patient nest, collectively input multiple data packets that each record the occurrence of at least one of the WHO's 5 Moments Hand Hygiene events.

The method of any of the preceding statements save, wherein multiple HCWs utilizing multiple types of hand hygiene dispensers can substantially simultaneously, within a single patient nest, collectively input multiple data packets that each record the occurrence of at least one of the WHO's 5 Moments Hand Hygiene events.

While the invention has been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

1. An apparatus for providing hygiene within a patient zone of a health care facility, comprising:
a mechanical connector having two ends;
a dispenser of a cleaning medium attached to an end of said connector; and
means for suspending the other end of said connector from a surface of the facility that is proximate to the patient;
wherein said dispenser is vertically suspended within the patient zone.

2. The apparatus of claim 1 wherein the surface is a ceiling.

3. The apparatus of claim 1 wherein the surface is a vertical wall.

4. The apparatus of claim 1 wherein the surface is part of a bed in the patient zone.

5. (canceled)

6. The apparatus of claim 1 wherein said dispenser requires physical motion by the worker to dispense the cleaning medium.

7. The apparatus of claim 1 wherein said connector is flexible between the two ends.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. An apparatus for providing hygiene to a health care worker within a patient zone of a health care facility, comprising:
a source of a cleaning medium;
a dispenser in fluid communication with said source and located within the patient zone, said dispenser including a dispensing nozzle; and
a plurality of marked actuators on said dispenser, said dispenser providing a quantity of the medium from said nozzle in response to actuation of any said actuator by the worker;
wherein each said actuator is marked differently than each other said actuator and each marking pertains to a different action by the worker chosen from a plurality of predetermined actions by the worker within the zone.

66. The apparatus of claim 65 wherein the predetermined actions include at least two of the Hand Hygiene events established by the World Health Organization.

67. The apparatus of claim 65 wherein the predetermined actions include before touching of the patient by the worker and after touching of the patient by the worker.

68. The apparatus of claim 65 wherein the predetermined actions include before the worker performs a procedures on the patient and after the worker has been exposed to body fluid of the patient.

69. (canceled)

70. (canceled)

71. (canceled)

72. (canceled)

73. The apparatus of claim 65 which further comprises a housing, wherein said source, said dispenser, and said actuators are contained by said housing, and said dispenser is electronically actuated.

74. A method for providing hygiene to a health care worker in a patient zone of a health care facility, comprising:
providing a computer and an electronic dispenser of a cleaning medium, the dispenser being located in the patient zone and having a plurality of actuators, each actuator having an annunciator, the computer being in electrical communication with the dispenser;
programming the computer with a list of predetermined actions, each action involving both the patient zone and the health care worker;
identifying each actuator with a different action chosen from the plurality of predetermined actions;
triggering the annunciator of at least one actuator by the computer; and
turning off the one annunciator in response to actuating the annunciated actuator.

75. (canceled)

76. (canceled)

77. (canceled)

78. (canceled)

79. (canceled)

80. The method of claim 74 wherein the computer is located proximate to the patient zone, the computer is operably connected to the dispenser, and which further comprises operating the dispenser by the computer to provide an amount of the medium in response to said actuating.

81. The method of claim 80 wherein the computer is separated apart from the dispenser and in wireless electrical communication with the dispenser.

82. (canceled)

83. The method of claim 74 wherein the computer is located remotely from the patient zone, and wherein said providing includes an electronic controller operably connected to said dispenser and in electronic communication with the computer.

84. The method of claim 83 which further comprises transmitting by the computer to the controller a signal corresponding to the list of predetermined actions.

85. The method of claim 83 which further comprises transmitting by the controller to the computer a signal corresponding to said actuating.

86. The method of claim 85 which further comprises preparing a data record by the computer of each said actuating.

87. (canceled)

88. (canceled)

89. The method of claim 74 which further comprises reporting each said actuating to the computer.

90. (canceled)

91. The method of claim 74 wherein said triggering is in response to the worker entering the patient zone.

92. The method of claim 91 wherein said providing includes an electronically actuated identification badge, wherein said triggering is in response to a signal from the badge.

93. The method of claim 91 wherein the dispenser includes a sensor that senses the proximity of the worker, and said triggering is in response a signal from the sensor.

94. (canceled)

95. (canceled)

96. (canceled)

97. The method of claim 74 wherein one of the predetermined actions is for the worker to clean hands before touching the patient, another of the actions is for the worker to clean hands after touching the patient, and the one action annunciates one actuator, and the other action annunciates another actuator.

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)
